(19) United States
(12) Patent Application Publication
(10) Pub. No.: US 2024/0044684 A1
(43) Pub. Date: Feb. 8, 2024

(54) DEVICE FOR MEASURING RATE OF BODY FLUID FLOW THROUGH A TUBE

(71) Applicant: Carilion Clinic, Roanoke, VA (US)

(72) Inventors: Mark Witcher, Boones Mill, VA (US); Connor Hale, Pulaski, VA (US)

(21) Appl. No.: 18/382,333

(22) Filed: Oct. 20, 2023

Related U.S. Application Data
(63) Continuation-in-part of application No. 18/138,022, filed on Apr. 21, 2023.
(60) Provisional application No. 63/333,634, filed on Apr. 22, 2022.

(51) Int. Cl. G01F 1/688; A61B 5/026

(52) U.S. Cl. CPC G01F 1/688; A61B 5/026

(57) ABSTRACT

An implantable flow meter device for measuring flow rate of a body fluid through a tube, such as a catheter and/or a ventriculoperitoneal (“VP”) shunt, comprising a flow meter attachment, in aspects attachable to pre-existing tubes, VP shunts, catheters, and/or peritoneal catheters systems, providing for the capability for physicians to obtain information about the status of the flow rate of the body fluid through the tube. The flow meter can include temperature sensors measuring the body fluid at a first and second position, as well as a heating element applying heat to the body fluid therebetween. A controller can receive the first temperature, the second temperature, and a heat value relating to the heat applied to the body fluid by the heating element, such that a processing device can calculate a flow rate of the body fluid based thereon.

### RELATED APPLICATIONS

The present application relies on the disclosure of and claims priority to U.S. patent application Ser. No. 18/138,022, filed Apr. 21, 2023, which claims priority to U.S. Provisional Patent No. 63/333,634, filed Apr. 22, 2022, the disclosures of which are incorporated by reference.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### FIELD OF INVENTION

The present invention relates generally to a catheter flow rate determination system and more specifically to determining a flow rate of cerebrospinal fluid (CSF) in a patient.

### BACKGROUND

The present invention is directed, in aspects, to an electromagnetic flow meter for diagnostic applications in ventriculoperitoneal (“VP”) shunts for treatment of hydrocephalus. Hydrocephalus is a condition characterized by excess fluid volume or pressure within the brain, which results in developmental, physical, and intellectual impairments. A cerebral shunt is typically used to treat hydrocephalus and remove excess fluid from the brain and relieve pressure, but there is no currently available device for monitoring a flow rate of the fluid to monitor proper function or malfunction. In aspects, the cerebral shunt consists of a ventricular catheter that is placed in the ventricles, a single-direction bi-compartmental valve, and peritoneal or other appropriate terminus-placed catheter. The disclosed ventricular shunt can have varying termini, including, but not limited to, peritoneum, atrium, pleura, amongst others that would be understood by one of skill in the art. The valve system function, in aspects, is for the physician to modulate the flow of cerebrospinal fluid (“CSF”) from the ventricles and into the receiving space, and can typically provide a reservoir for transcutaneous tapping. Tapping a shunt refers to the removal of fluid from the compartment via a transcutaneously-placed syringe for analysis of function, including flow as well as sampling of CSF. There are inadequate options on the market that inform the physician of the current flow rate of the CSF through the VP shunt and thus the functionality of the shunt is unknown until invasively monitored by medical personnel.

In aspects as described herein, an electromagnetic multi-magnet array is used to measure CSF (or other conductive fluid) flow based on the ionic properties of the CSF (or other conductive fluid). According to one potential embodiment, the magnetic field used within the device is generated by a Halbach Cylinder, a type of permanent magnet array wherein a velocity-dependent voltage is created via magnetic induction. (However, several other examples of magnetism and electromagnetism are contemplated other than the Halbach Cylinder.) In other embodiments, a magnet array is used that can mimic or replicate the effects of a Halbach Cylinder array, or otherwise provide a magnetic field that is directionally perpendicular to the flow of fluid inside a catheter. Magnet arrays can include, but are not limited to, bar magnets, ring magnets, premade halbach, custom halbach (oriented by user or manufacturer), and arc magnets. Further, in aspects, lead wires coming into contact with the fluid (e.g., CSF) are configured perpendicular to the direction of the magnetic field. The magnetic flow meter is created to be in-line with the peritoneal (or other terminus) catheter to prevent additional risks of clogs and be compatible with standard peritoneal catheters. The voltage is then conditioned for wireless transmission to a remote electronic device, including but not limited to a smart phone, pager, pager-like device, computer, laptop computer, tablet computer, computer processor, storage device, etc. The collection and relaying of this information allows for physicians to be able to determine when the shunt malfunctions without the need for invasive methods.

Hydrocephalus is an excess buildup of CSF pressure or volume within the brain, specifically in the cerebral ventricles. This fluid causes the ventricles to expand, which places pressure on the brain's tissues. The two major types of hydrocephalus are communicating hydrocephalus and non-communicating hydrocephalus. Hydrocephalus can develop at any age, but is found to be more common in infants and adults that are 60 and older. Typical symptoms of hydrocephalus include headache, blurred vision, difficulty walking, neck pain, and confusion.

If it is determined that the patient has hydrocephalus, many patients are recommended a VP shunt as the standard of care, which shunts CSF from the ventricles to a selected terminus, varying in destination from patient to patient. This technique has been generally an effective solution to hydrocephalus, although common complications include blockage of flow and infection, both of which can have significantly morbid consequences, including extended hospitalizations, repeat surgeries, and death.

Monitoring of flow rate according to the present invention, can allow earlier notifications of decreased or cessated flow, allowing earlier, proactive interventions. Without monitoring, the shunt blockage only becomes apparent after symptoms return to the patient. Currently, only one development for shunt flow rate has become commercially available, but it relies on temperature variations of the CSF generated ex vivo (outside the body). (See, ShuntCheck™, https://neurodx.com/shuntcheck/.) In the case of ShuntCheck™, temperature sensors are placed external to the shunt to record temperature variations; in other words, the system cools the skin and then measures the rewarming based on CSF flow. However, the device overall is often difficult to use and can provide inaccurate results, as it is not self-monitoring and, again, the measurements occur ex vivo. Accordingly, there is a long-felt need for a system/device to monitor a VP shunt/catheter treatment system.

In aspects, the invented device described in the current Application uses an electromagnetic flow meter similar to that described by Vennemann et al. (See, Vennemann B, Obrist D, Rösgen T (2020) A smartphone-enabled wireless and batteryless implantable blood flow sensor for remote monitoring of prosthetic heart valve function. PLoS ONE 15(1): e0227372. https://doi.org/10.1371/journal.pone.0227372)

There, the design group developed a cusp-like electromagnetic flow meter intended to be secured to the ascending aorta. The current device described herein is different in many regards and an innovation and improvement over Vennemann et al. Their design is developed to measure blood flow in a blood vessel and measures voltages through the vessel wall rather than with direct contact with the fluid.

Moreover, the quantity of fluid measured Vennemann et al. may greatly differ compared to what can be measured by the current invention (e.g., on average aorta flowrate is around 200 ml/sec compared to a CSF target flow rate of around 0.5-1.0 ml/min.) Regarding the conductivity of blood compared to CSF, blood is less conductive than CSF.

### BRIE DESCRIPTION

As described herein, in aspects, a flow meter is capable of determining the flow rate of CSF (or other conductive body fluid). In an embodiment, the flow meter may operate within or in conjunction with a catheter system, allowing for more precise adaptations to be made to the flow rate based on the current needs of the patient. The amount of CSF produced and traveling through the catheter changes based on several factors and determinants, and thus requires a system that can allow for continuous monitoring to facilitate needed adaptations.

In an embodiment, the flow meter is a medical device that measures the flow of CSF through a ventriculoperitoneal shunt by measuring the temperature of liquid, applying heat to the liquid, and then measuring the new temperature. In aspects, it is a small amount of heat applied to the liquid, such as between about 1 and about 5 degrees Fahrenheit, by way of example only, such as between 0 and 1 degree, 1 degree and 2 degrees, 2 degrees and 3 degrees, 3 degrees and 4 degrees, and 4 degrees and 5 degrees. Herein, a flow rate can be calculated by measuring the variable heat differential, or in another embodiment, by measuring an amount of power required to maintain a constant heat differential.

Heat measurement can be performed using any number of suitable processing techniques. For example, embodiments can include measuring heat using one or more thermistors, heat sensitive resistors, thermocouples, silicon-based temperature sensors, or any other suitable tool(s) for temperature measurement. The heat applied to the CSF can be generated using any suitable heating system. Embodiments can include using an inductive heating system, conducting heating elements, resistive heating coils, and/or heat-generating thermistors, by way of example.

In a further embodiment, the medical device can include a communication system for transmitting acquired data. The communication system can transmit the measurement information for external processing. The communication system can transmit locally-processed data and value. In an embodiment, the communication system can transmit data to doctors or other healthcare professionals, providing the CSF flow rate for improving patient care. The transmission system can transmit data using any suitable transmission technique, including for example radio-frequency transmissions, wireless transmissions, e.g. Bluetooth® or other suitable wireless techniques, Wi-Fi frequency band transmissions, or any other transmission path as recognized by a skilled artisan.

As described in greater detail below, when associated with an implanted medical device, e.g., a shunt, the transmission system may include the transmitter disposed within the implanted medical device, operating in combination with an external receiver having a display and user interface. For example, one embodiment may include an implanted transmitter with a receiver in the form of a computing device such as a phone, tablet computer, laptop computer, desktop computer, mobile computing device, server, dedicated hardware device, etc., in wireless communication via the transmitter.

A further embodiment of the device can include a power system for long-term use within the patient. The power system can include a variety of different embodiments, providing not only for distribution of power, but also techniques for recharging for the long-term use. In one embodiment, the power may be transmitted wirelessly through the use of inductive coils or RF power transfer. In another embodiment, power can be generated based on temperature differentials within the body of the patient or in another embodiment movement of the patient. This power can be stored and/or dispensed for operations as noted herein.

The device is not limited by the patient's body composition, as the wirelessly transmitted data can be communicated through all tissue types. The system is able to transmit data from patients through multiple BMI ranges, comorbidities, and outstanding conditions.

This technology can also determine the condition of the shunt, by measuring flow and indicating when flow is decreased or stopped. A change of flow can assist physicians with determining failure of the shunt and better predict if a surgical intervention will be needed.

It is an object of the present invention to provide noninvasive feedback to both patients and their medical team on a regular and/or continuous basis, depending on the needs of the patient. This feedback can include, but is not limited to, information on the flow rate and/or the current functionality of the device. It is also contemplated that the feedback can include possible evidence of infection, as infection can sometimes decrease the flow of CSF through the shunt. For example, bacterial infections can raise protein and lower glucose, as well as produce lactate in the CSF, which may change the ionic charge of the CSF, slow CSF flow, or otherwise indicate to the system described herein of the possibility of an infection(s). The device may also allow for adjusting of flow rate without requiring surgery, as a patient's needs from the device can change over time. In aspects, the device can be added to existing shunt devices, as removal of a currently installed shunt can cause unnecessary physical trauma to the patient. In embodiments, the intended use of this device is as a long-term treatment for patients with hydrocephalus by draining CSF from the ventricles in the brain to the peritoneal space to reduce pressure and long-term stress on the brain.

In embodiments, the device is an electromagnetic or temperature-based flow meter that is designed to be integrated into VP shunt systems, which typically include a ventricular and peritoneal shunt separated by a valve. In aspects, the flow meter could be applied downstream from the valve on the distal catheter. As CSF flows through the valve and into the peritoneal catheter, the CSF reaches laminar flow and feeds into the uniform magnetic field created by the flow meter. In aspects, it is envisioned that aspects of the current invention could be used on the cranial side of the shunt. In embodiments, the device, including the electromagnetic or temperature-based flow meter, as described herein, could be added anywhere along the length of a shunt, such as a VP shunt. This field encourages charged ionic particles to separate proportional to fluid velocity, creating a voltage differential. Electrodes set in the wall of the flow meter in contact with the fluid measure this voltage. A signal processor implanted in conjunction with the flow meter can be used to perform signal conditioning to translate the signal into a wirelessly transmissible form with the intention to relay the information out of the body. In aspects, a collection device carried with the patient will communicate with the implanted device on a regular or continuous basis and store the flow meter information. This device may also upload the information to a location accessible by the patient, confidants of the patients, the patient's physician, along with others permitted to see that information.

In aspects, inductive coils can be used to transmit power to the electromagnetic or temperature-based flow meter. A connected batter can also be used In aspects, shielding can be used to contain or otherwise influence the electromagnetic field. In aspects, a separate connection without a magnet is used for grounding to generate a reference point for measurements.

### BRIEF DESCRIPTION OF THE DRAWINGS

The accompanying drawings illustrate certain aspects of some of the embodiments of the present invention, and should not be used to limit or define the invention. Together with the written description the drawings serve to explain certain principles of the invention.

FIG. 1 is a table depicting that, in aspects, CSF flow through a distal catheter is laminar.

FIG. 2 is a graph showing validation that the magnetic field generated by the Halbach cylinder in the 7:1 scale embodiment dissipates outside of the array. There is no measurable magnetic field past 4 mm in this particular model.

FIG. 3 is a graph showing a similar trend as in FIG. 2. In the 4:1 scale embodiment depicted, the magnetic field dissipates outside of the Halbach array. There is no magnetic field detected 3 mm radially away from the 4:1 embodiment in this particular example.

FIG. 4 is a graphic showing the operating principle of the Halbach array specific to a particular embodiment's design. Included is the equation for voltage generation as a function of CSF flow and placement of the magnets and electrodes.

FIG. 5 is an image showing the experimental setup for the 4:1 embodiment flow test.

FIG. 6 is a graph showing measured voltage versus flow rate, in an embodiment. Each value of conductivity includes three trials in this example.

FIG. 7 is a depiction of a magnetic field generated by 10 magnets as predicted by COMSOL Multiphysics Software.

FIG. 8 is a depiction of a magnetic array as generated by COMSOL without the center field showing as in FIG. 7.

FIG. 9 is a depiction of the Halbach cylinder with barb connections included. The embodiment in this example assumes electrical components will be placed in the bottom of the device.

FIG. 10 depicts an embodiment with adjusted barb connectors for the peritoneal catheter. This embodiment assumes electrical components will be placed adjacent to the Halbach cylinder.

FIG. 11 is a schematic showing similar barbs to FIG. 9. This embodiment assumes the electronics will be placed along the cylinder.

FIG. 12 is a graphic showing a possible Halbach array embodiment.

FIG. 13 is a flowchart of one possible non-limiting embodiment of the current invention.

FIG. 14 is a morphological chart showing one possible non-limiting embodiment of the current invention.

FIG. 15 is a graphic showing a possible Halbach array embodiment.

FIG. 16 shows a housing for the electromagnetic array according to an embodiment of the invention.

FIG. 17 shows a housing for the electromagnetic array according to an embodiment of the invention.

FIG. 18 shows an embodiment of the current invention wherein direction of fluid flow and location of leads are perpendicular to the magnetic field direction.

FIG. 19 is an overall system diagram according to an embodiment of the present invention.

FIG. 20 shows an outline of the two different arrays according to embodiments of the present invention; array 1 being magnetized and array 2 not being magnetized. Array 1 does not have to be before Array 2, in aspects.

FIG. 21 illustrates a block diagram of one embodiment of a device for measuring fluid rate.

FIG. 22 illustrates a block diagram of an embodiment of a controller of FIG. 21.

FIG. 23 illustrates a depiction of a device according to embodiments described herein.

FIG. 24 is a chart describing characteristics of the invention according to embodiments described herein.

FIG. 25 shows the data used to create the chart in FIG. 24.

### DETAILED DESCRIPTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

### The Device

A need exists for a VP shunt to include the ability to perform one or more of the following functions: measure flow-rate at regular or continuous intervals, provide feedback on correct placement of the shunt, allow for adjustable flow rate, enhance durability, provide accessibility for all body types, and contain an information relay that transmits to a device outside a patient's body. As described herein, embodiments of the current invention include a device that is compatible with current catheters and shunts on the market.

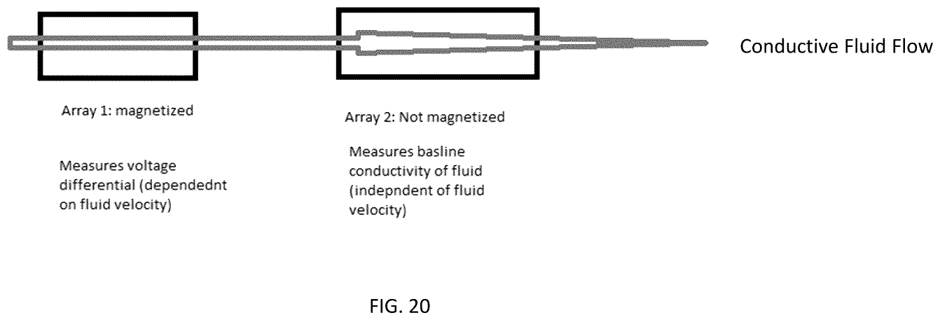

In embodiments, the device comprises an electromagnetic flow meter including, in aspects, several components. The first is a magnet array and flow fixture. This component, which in aspects is the largest of the components, comprises the meter itself where CSF (or other conductive fluid) flows through the fixture and a voltage is generated proportional to the velocity of the fluid. In embodiments, a second plurality of electrodes can measure voltage without the magnet array to establish a baseline for measurements and check for infection. (See, FIG. 20.) Another component is the signal conditioner and antenna. Here, the raw signal can be conditioned so that it can be transmitted via, in aspects, RFID or other form of short range wireless communication. Another component is the receiver, which may be a phone or stand-alone device that receives the data and is accessible to the patient or medical providers. It also can be a device mounted on the body and can contain the power source and inductive coils for power and/or transmission. In embodiments, the device may use RF to DC or NFC (near field communication) power.

In aspects, the overall device also includes a means for providing power to the electromagnetic flow meter, such as a battery or wireless power transmission using, for example, inductive charging coils.

With respect to the Halbach Cylinder, which is envisioned in embodiments, it can be a circular array of permanent magnets that produce distinct magnetic field patterns inside and outside the cylinder. In aspects, for the purpose of a magnetic flow meter, the Halbach Cylinder can produce a uniform magnetic field across the interior of the cylinder. Due to the precise and intentional orientation of the magnets, a negligible field is produced outside of the Halbach Cylinder. In other embodiments, a magnetic field can be produced to monitor flow using a series of electrically conductive coils. When wound in a circle with current passing through the coils, a uniform magnetic field is created around the circumference of the coils. The magnetic field created is not contained within the coils and a substantial external field is created. In embodiments, the magnetic field generated by the coils can be uniform with respect to position, but could also be time-varying.

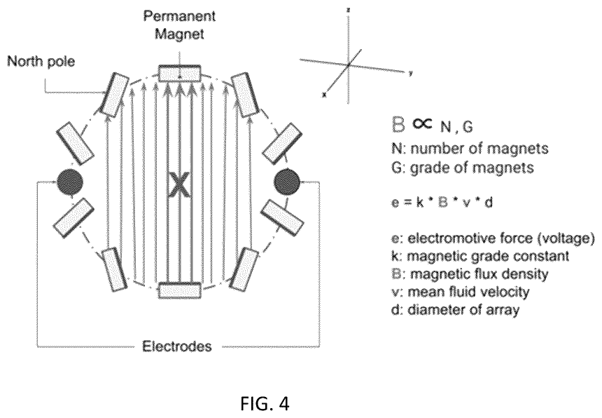

In aspects, a catheter that is transporting CSF from the ventricles to, for example, peritoneal space can be routed through the center of the modular Halbach Array Cylinder, as shown in, e.g., FIG. 4. Tangential to the plane of fluid flow and the field lines of the magnetic array, there can be a porthole for electrical components to be attached to or otherwise connected to the array. This port can be placed tangential to both flows in order to maximize the voltage potential created by the CSF flow and Halbach Array interaction. The conductivity of the flowing CSF through the uniform magnetic field generates a voltage normal to both the field and flow. This voltage generated is the raw signal, which can be conditioned and reported to the physician.

In aspects, devices described herein do not cause a noticeably larger or substantially larger form factor compared to current shunts. The devices described herein can be compatible with patients of many, most, or all BMI ranges. The devices described herein can be used as an attachment to current VP shunts, meaning that for users that already have a VP shunt implanted, the device described herein can be attached or otherwise connected to their current shunt and/or catheter instead of getting an entirely new VP shunt. This also applies for current VP shunts that have already been manufactured.

The devices described herein help monitor the functionality of the VP shunt, which can allow the existing shunt and/or catheter to be better or more easily monitored and maintained over time, which can result in less failures and shunt/catheter replacement.

### Related Process

In embodiments of the current invention, a trigger signal can be sent wirelessly to the implanted flow monitoring devices as described herein. In aspects, communications can be initiated by the implanted devices described herein. The devices described herein can make use of RFID, near field communication, bluetooth, or other methods for data transmission. Sources of this signal could be encrypted in order to ensure safe and private use of the system for the patient. Sources of this signal could, in aspects, be cell phones, a specifically designed device the size of a cell phone or smaller that could query the meter and collect the data, and/or a computer or computer processor, by way of example only.

Once the trigger signal is received, conditioning may be required, such as for example using a preprocessing system, which can include an antenna and a mechanism for converting the radio waves to an electrical signal that can be used by the actual meter. One or more sensors in or connected to the implanted device can generate a property-dependent electrical signal. This measurement on the fluid velocity can be performed using the given design's exploiting property, and thereby generating its property-dependent electrical signal. From there, post-processing can be used to define resolution and range, as well as prepare the signal to be transmitted to the data collector. The data output, in aspects, can also be encrypted in order to protect the user's privacy. In aspects, this process occurs continually, in intervals, and/or upon command. In aspects, the process and measurements can happen in real-time or near real-time, such as, by way of example only, 0-1 second, 1-2 seconds, 2-3 seconds, 3-4 seconds, and so on. Faster runtimes can allow for measurements to be taken in more rapid succession, resulting in more informed patients and medical providers.

In embodiments, because the devices described herein are measuring an extrinsic property, flow needs to be uninterrupted. This can be fulfilled by building the devices described herein as an inline attachment to the catheter. Furthermore, in aspects, no power needs to be supplied, as a goal is to observe the natural state of the CSF flow. This simplifies the material input and power supply for decomposition. This leaves, in aspects, signal management to be the central component of the decomposition.

### Test Results for Electromagnetic Embodiments

To determine an efficient and safe number and positioning of magnets in the Halbach Array, several arrays were tested using COMSOL multiphysics simulation software. A preliminary study was conducted first to evaluate the differences between cylindrical and rectangular magnets. This was done using two sets of 12 N42⅜″ permanent magnets oriented such that two revolutions were completed within the Halbach Array. The tests run were parts of the AC/DC module and included Magnetic Fields with No Current and Magnetic Field packages.

A finer mesh was applied to the magnets and an air domain was included to visualize and quantify the magnetic field lines and magnetic flux of the array. When the two arrays were compared, it was determined that, in aspects, the shape of the face of the magnet does not influence the magnetic field line paths, density, or strength: both magnetic fields studied were at a peak of 1.1 mT within the array and rapidly approached zero outside of the array. In aspects, rectangular magnets are preferred, given that the rectangular magnets are less prone to rolling and easier to manipulate during manufacturing and fixation to the testing array component. Further, when considering the possibility of stray field lines, rectangular magnets did not experience this phenomenon. However, differently shaped magnets can be used, such as circular, ovular, tubular, hexagonal, polygonal, square, rounded, cylindrical, spherical, triangular, and other shapes.

Following this preliminary study, additional computational results were run to validate the extent and limitations of the magnetic array at generating a concentrated magnetic field that does not deviate from the limits of the array. The purpose of this model was to ensure that the magnetic field was appropriately contained within a small model. This was accomplished by using a set of 12 N42 cubic magnets, oriented such that two revolutions were completed within the Halbach Array. The tests run were parts of the AC/DC module and included Magnetic Fields with No Current and Magnetic Field packages. A finer mesh was applied to the magnets and an air domain was included to represent the magnetic flux and field lines. Despite the small radius, the magnetic field lines remained within the Halbach Array and the magnetic flux varied only slightly by less than 10% difference between overall magnetic field strength.

Additional computational models were performed to a potential number of magnets required to generate a constant magnetic field with desired magnetic flux and strength. In other words, to determine the amount of magnets required to create a constant field in order to minimize the amount of magnets needed as a result of a space and size constraint. In order to do this, two simulations were run using the Magnetic Field with No Current and Magnetic Field packages in COMSOL. The studies determined that between 10 and 14 magnets there was less than a 10% difference between magnetic flux. In order to keep the Halbach Cylinder suitably sized for the application described herein, a 10 magnet array instead of a 12 or 14 magnetic array could be used, although other numbers and sizes are contemplated, including more than 10 magnets and less than 10 magnets. The unique and intentional spacing of the magnets provided by the 10 magnet Halbach Array allows for placement of electrodes and/or other components of the device tangential to or otherwise distance-separated from both the magnetic field generated and the flow of the CSF through the catheter system. This placement can assist with gathering accurate data pertaining to the flow rate of the CSF.

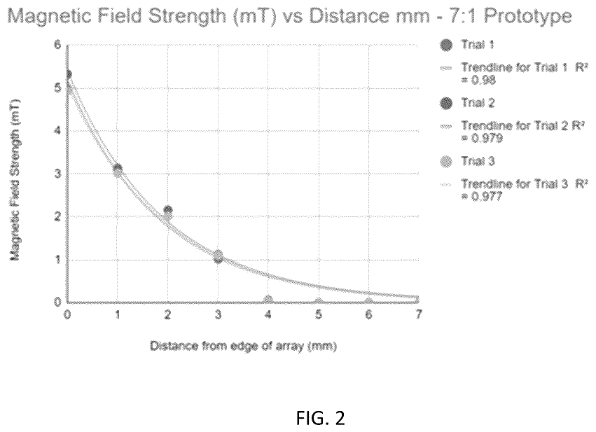

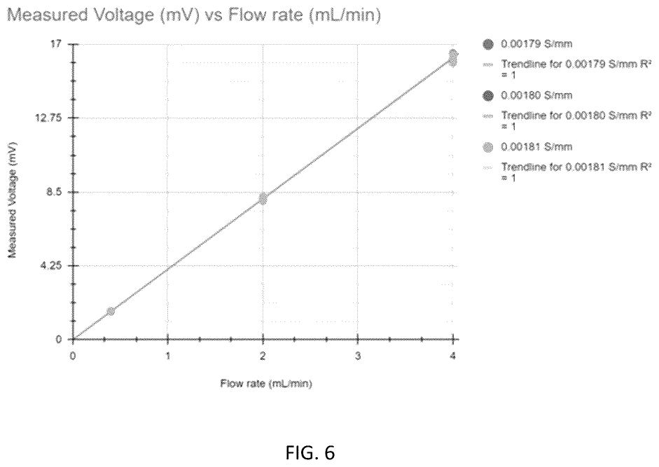

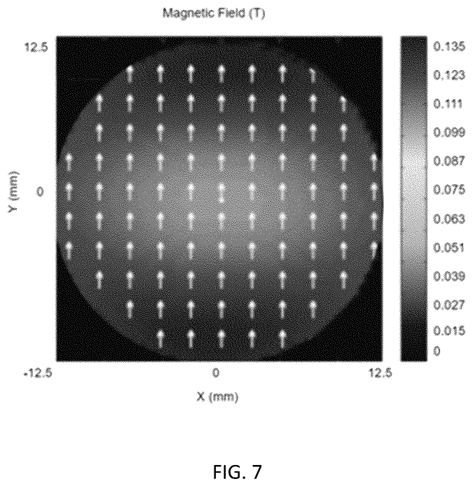

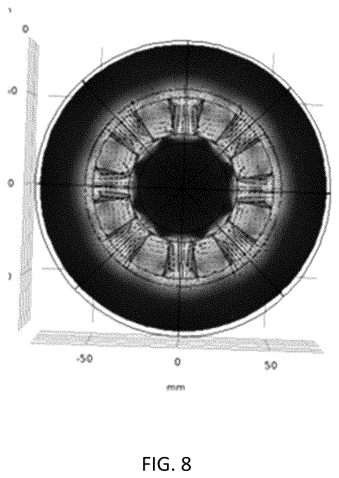

Studies were conducted in a bench-top setting to validate the previous models performed in COMSOL Multiphysics software. The experimental results can be found in FIGS. 2, 3, and 6. These results further validate the relationship between fluid flow and induced voltage as a result of CSF-analog fluid moving through the Halbach cylinder.

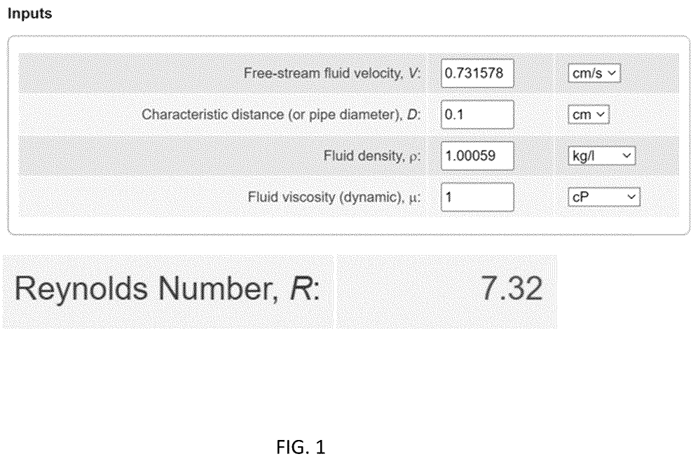

For the first test, a syringe was attached to a mechanical syringe depressor that determines the flow rate based on the volume of the syringe used and a rate of compression of the syringe plunger. The peritoneal catheter was attached to the end of the syringe. The device was turned on and began compression of the plunger at a flow rate similar/analogous to that seen in hydrocephalus patients. The catheter was observed to determine if flow rate is visible or measurable or detectable through the system in real-time or near real-time through the entire length. The flow was allotted 30 seconds to determine this. Flow was noted at the end of the catheter and continued through the entire duration of the volume of the syringe, 3 mL. The Reynolds number for the flow through the peritoneal catheter was 7.32 when measured 0.1 cm from the start of the catheter, making the flow both above the threshold for creeping flow (>1) and below turbulent flow (<1000), thereby laminar. This is demonstrated in, e.g., FIG. 1.

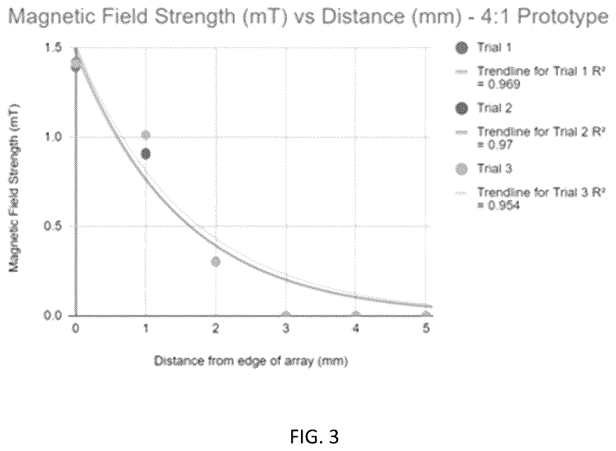

The purpose of the second test is to determine that there is no external magnetic field outside of the Halbach array, as suggested by the computational modeling done in COMSOL Multiphysics software. This is to determine that there is little/minimal to no risk of interaction with other implantable devices, external magnetic fields, or with the human body. The Halbach array was placed in an electromagnetically isolated environment and a Gauss meter was used to detect the remnant magnetic field outside of the array at varying distances. This study was conducted without the use of additional shielding to further validate the Halbach cylinder's ability to contain magnetic flux within the array. The results from this study verified the previous modeling done in COMSOL Multiphysics software and can be seen in FIGS. 2 and 3.

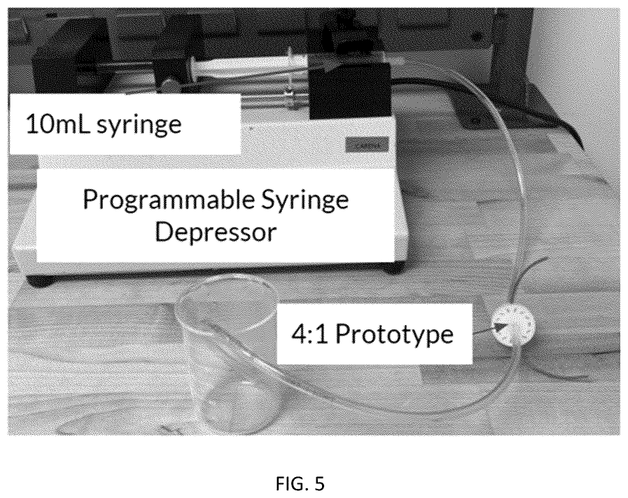

An additional test was performed to show that the device will be able to operate as described at different scales and varying parameters that are seen in the human body and within patients suffering from hydrocephalus. Both a large scale and small scale prototype were tested with 10 N48 sintered magnets arranged into a Halbach array. The array was attached to a commercial pump that moved conditioned water from a reservoir through tubing into the array and returned to the reservoir. The water used was conditioned to match ranges of conductivity seen in CSF, with ranges from 1.79 S/m to 1.81 S/m. The voltage was measured using an electrode placed into the side of the catheter connected to a voltmeter that displayed the induced voltage in real-time. Five trials were conducted to gather data points that are analyzed in, e.g., FIG. 5.

Turning to the other Figures, FIG. 4 demonstrates the operating principles of the magnetic flow meter using the 10 magnet Halbach array. Lorentz law is shown only to describe the electromagnetic induction as a result of laminar and conductive/ionic fluid flow (CSF) through the uniform magnetic field.

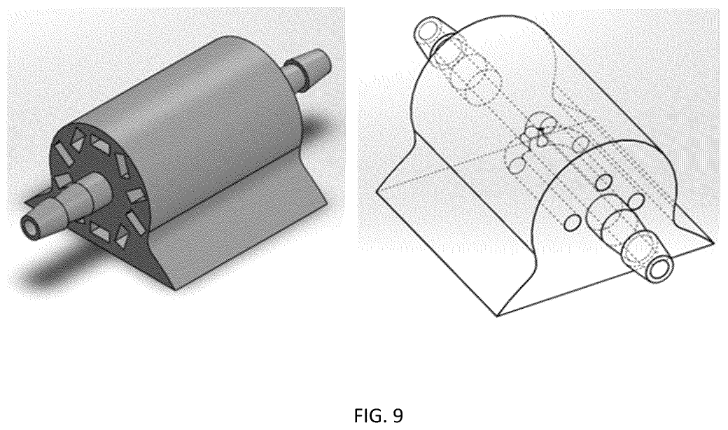

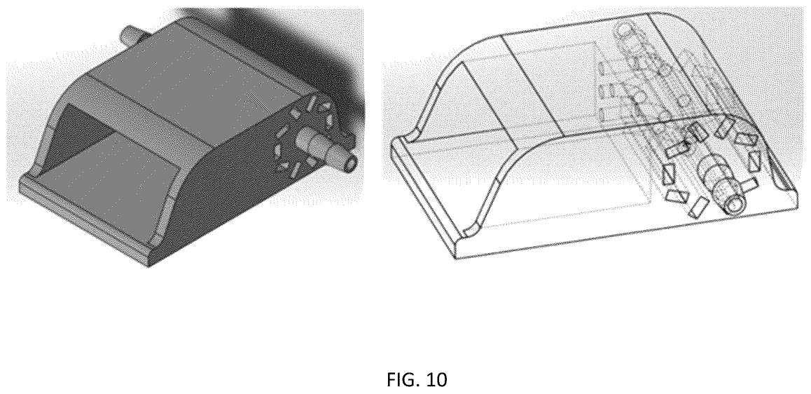

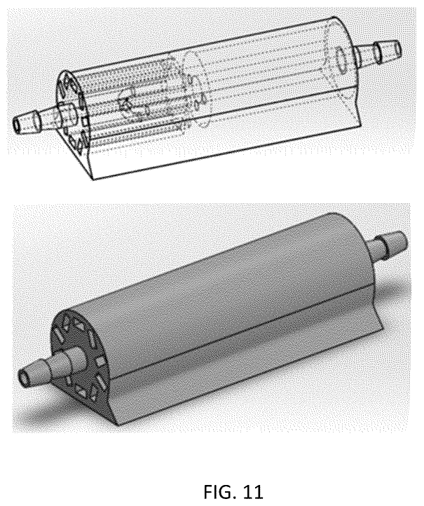

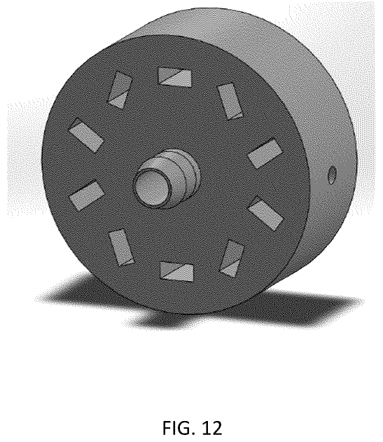

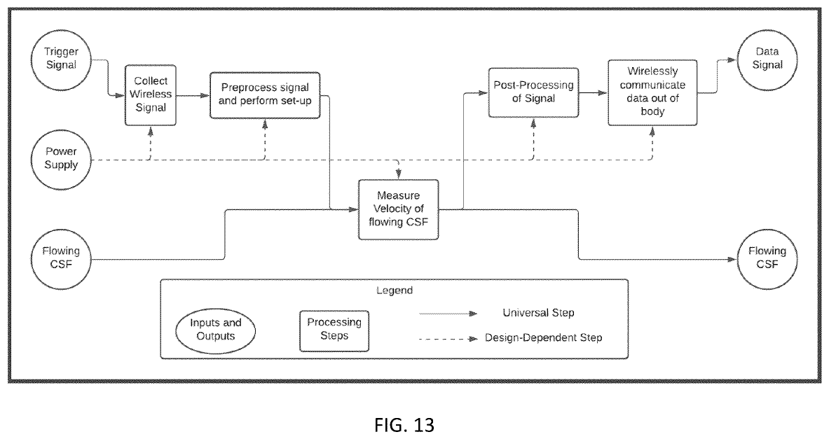

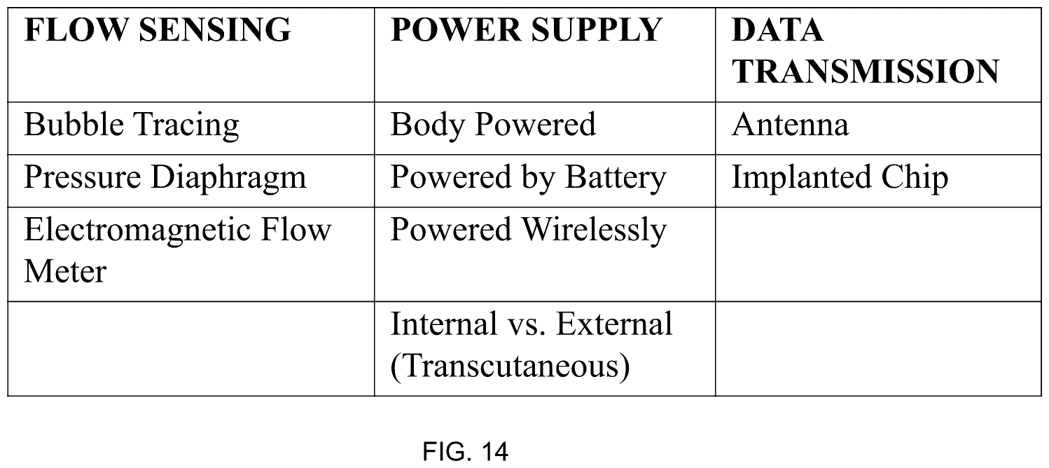

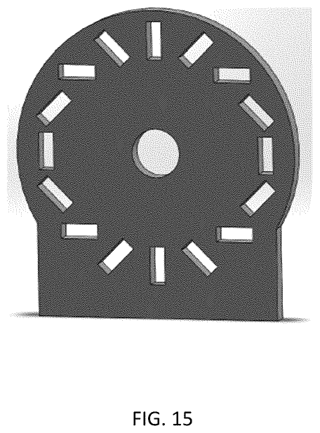

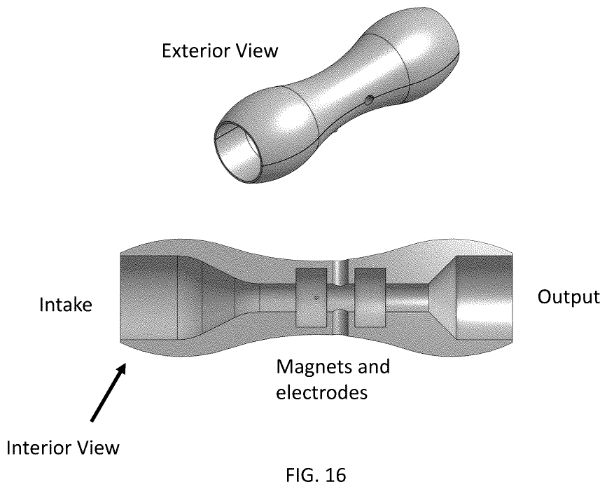

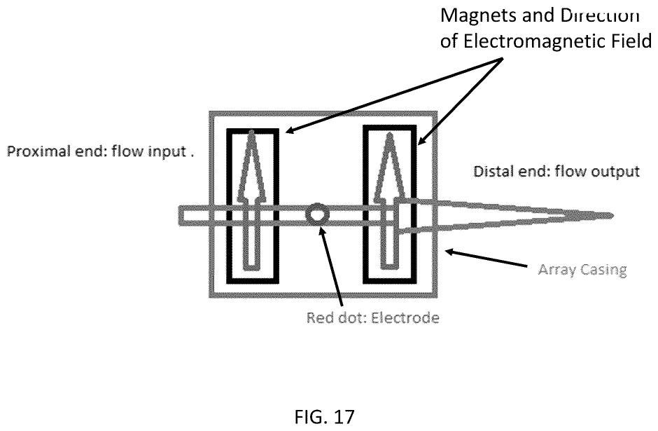

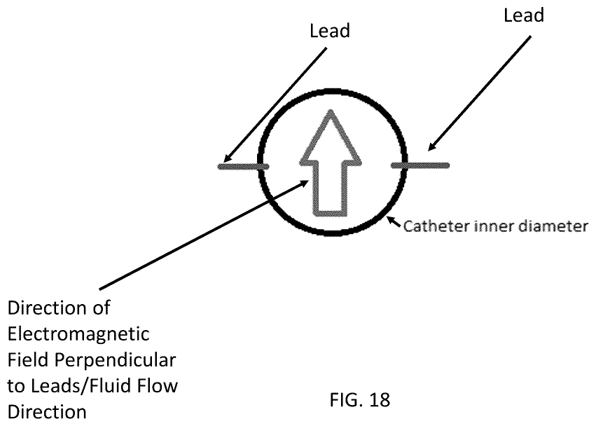

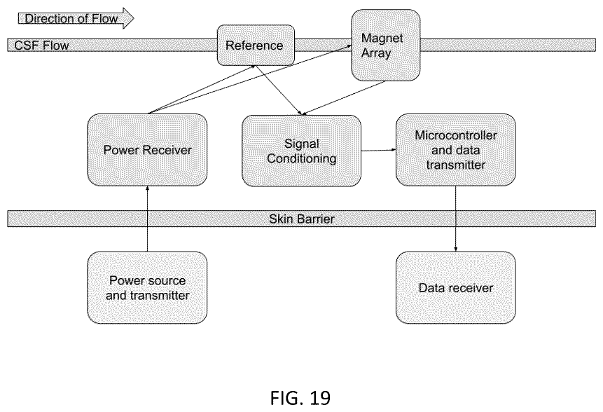

FIGS. 9-11 are related to the construction of the mountable meter itself. Several designs are contemplated, as described herein. FIG. 9 shows the entire flow meter and connecting barbs with the proposed electronics being placed in the bottom of the mountable portion, while FIG. 10 shows it being placed to the side, and FIG. 11 to be placed radially.

Embodiments of the invention also include a computer readable medium comprising one or more computer files containing applications, frameworks, libraries, and such, comprising a set of computer-executable instructions for performing one or more of the calculations, steps, processes and operations described and/or depicted herein. In exemplary embodiments, the files may be stored contiguously or non-contiguously on the computer-readable and/or device-readable medium. Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), memory card, and RAM. In exemplary embodiments, the computer readable medium has a set of instructions stored thereon which, when executed by a processor, cause the processor to perform tasks, based on data stored in the electronic database on the computer or cloud, or memory described herein. The processor may implement this process through any of the procedures discussed in this disclosure or through any equivalent procedure.

In other embodiments of the invention, files comprising the set of computer-executable instructions may be stored in computer-readable memory on a single computer or distributed across multiple computers, in personal communication device and/or devices, or be stored in cloud computer. A skilled artisan will further appreciate, in light of this disclosure, how the invention can be implemented, in addition to software, using hardware or firmware. As such, as used herein, the operations of the invention can be implemented in a system comprising a combination of software, hardware, and/or firmware.

Embodiments of this disclosure include one or more computers or devices loaded with a set of the computer-executable instructions described herein. The computers or devices may be a general purpose computer, a special-purpose computer, personal communication device, or other programmable data processing apparatus to produce a particular machine, such that the one or more computers or devices are instructed and configured to carry out the calculations, sensor data collecting and processing, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (e.g., processor or System on Chip (“SOC”)) and a form of computer-readable memory which may include random-access memory (“RAM”) or read-only memory (“ROM”). The computer-executable instructions can be embedded in computer hardware or stored in the computer-readable memory such that the computer or device may be directed to perform one or more of the calculations, steps, processes and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system can include a stand-alone computer, such as a desktop computer, a portable computer, such as a tablet, laptop, PDA, wearable device (e.g., electronic watch, smart glasses or HMD—Head Mounted Display), or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

The input/output user interfaces may include a graphical user interface (GUI), voice command interface, gesture interface, gaze interface, or combinations thereof, which may be used in conjunction with the computer-executable code and electronic databases. The graphical user interface gesture interface, gaze interface, or combinations thereof, may allow a user to perform these tasks through the use of text fields, check boxes, pull-downs, command buttons, voice commands, various gestures gaze as a selection mechanism, and the like. A skilled artisan will appreciate how such user features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection.

In embodiments, an invention described herein includes several Aspects, including:

Aspect 1: An electromagnetic flow meter for determining a flow rate of a conductive fluid, the electromagnetic flow meter comprising the following:
a magnet array comprising at least two magnets providing an electromagnetic field direction perpendicular to a flow direction of the conductive fluid;
a plurality of electrodes positioned perpendicular to both the electromagnetic field direction and the flow direction of the conductive fluid;
wherein the flow of the conductive fluid through the electromagnetic field generates an induced voltage;
wherein a measured induced voltage is proportional to a conductive fluid velocity, thereby providing a velocity-dependent voltage differential;
wherein the velocity-dependent voltage differential is measured by the plurality of electrodes to determine the flow rate of the conductive fluid.

Aspect 2: The electromagnetic flow meter of Aspect 1, further comprising a second plurality of electrodes located at a location separate from the magnet array, wherein the second plurality of electrodes measures a conductivity of the conductive fluid to provide a reference point for the flow rate determination.

Aspect 3: The electromagnetic flow meter of Aspect 1, further comprising a second plurality of electrodes located at a location separate from the magnet array, wherein the second plurality of electrodes measures a change in the conductivity of the conductive fluid over time to determine if an ionic change to the conductive fluid has occurred.

Aspect 4: The electromagnetic flow meter of Aspect 3, wherein the change in the conductivity of the conductive fluid indicates an infection or other fluid pathology.

Aspect 5: The electromagnetic flow meter of Aspect 1, wherein the electromagnetic flow meter is at least one of attached to, integrated with, or in-line with, a shunt.

Aspect 6: The electromagnetic flow meter of Aspect 1, further comprising a transmitter capable of sending data from the electromagnetic flow meter to a remote electronic component configured to receive the data from the electromagnetic flow meter.

Aspect 7: The electromagnetic flow meter of Aspect 1, wherein a remote electronic component is configured to communicate with the electromagnetic flow meter to receive flow rate information from the electromagnetic flow meter.

Aspect 8: The electromagnetic flow meter of Aspect 1, wherein a remote electronic component is configured to transfer power to the electromagnetic flow meter.

Aspect 9: The electromagnetic flow meter of Aspect 1, wherein the electromagnetic flow meter is connected to a shunt and a bi-compartmental valve, wherein the bi-compartmental valve acts to modulate the flow rate of the conductive fluid from a first location to a second location.

Aspect 10: The electromagnetic flow meter of Aspect 9, wherein the first location is one or more patient ventricle.

Aspect 11: The electromagnetic flow meter of Aspect 9, wherein the second location is a fluid receiver.

Aspect 12: The electromagnetic flow meter of Aspect 9, wherein the bi-compartmental valve comprises a reservoir for transcutaneous tapping.

Aspect 13: The electromagnetic flow meter of Aspect 9, wherein the shunt is a ventriculoperitoneal shunt.

Aspect 14: The electromagnetic flow meter of Aspect 9, wherein the shunt is a ventriculoperitoneal shunt for treatment of hydrocephalus.

Aspect 15: The electromagnetic flow meter of Aspect 1, wherein the conductive fluid is cerebrospinal fluid.

Aspect 16: The electromagnetic flow meter of Aspect 1, further comprising at least one catheter.

Aspect 17: The electromagnetic flow meter of Aspect 16, wherein the at least one catheter is a ventricular catheter.

Aspect 18: The electromagnetic flow meter of Aspect 16, wherein the at least one catheter is a peritoneal catheter.

Aspect 19: The electromagnetic flow meter of Aspect 17, wherein the ventricular catheter originates in a patient's ventricles.

Aspect 20: The electromagnetic flow meter of Aspect 9, wherein the bi-compartmental valve is a single-direction bi-compartmental valve.

Aspect 21: The electromagnetic flow meter of Aspect 1, further comprising a power source.

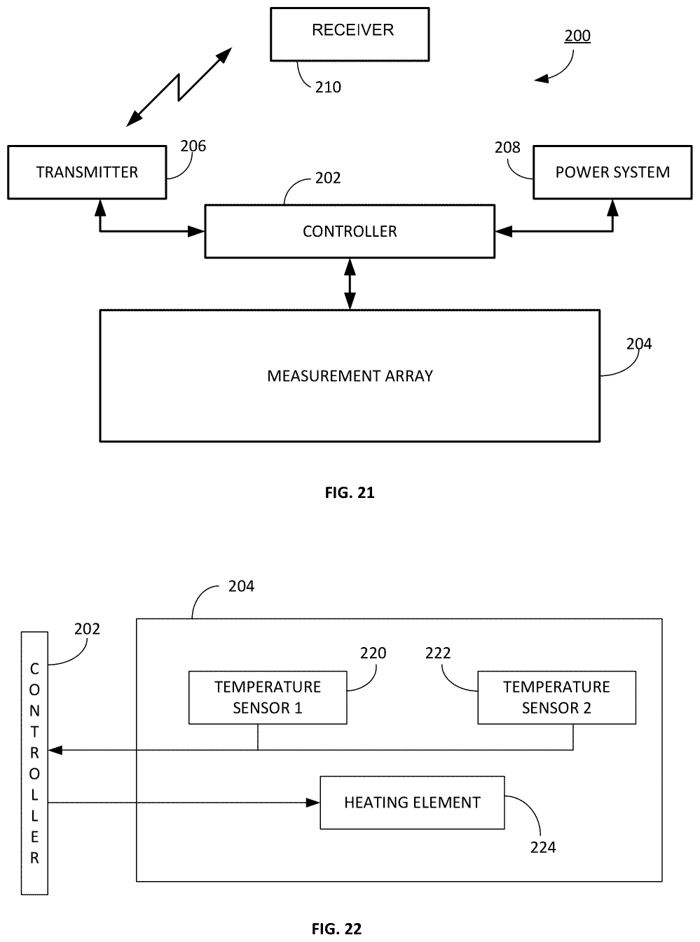

Aspect 22: The electromagnetic flow meter of Aspect 1, wherein power is provided to the electromagnetic flow meter wirelessly using inductive coupling or using wire with a subcutaneously implanted battery physically connected to and powering the electromagnetic flow meter.

Wherein the above Figures provide for varying embodiments of measuring CSF, FIG. 21 illustrates another embodiment including system 200. The system 200 includes a controller 202, a measurement array 204, a transmitter 206 and a power system 208. The system 200 may also include a receiver 210.

The system 200 may be embedded within a shunt of other system as described above. The system 200 may be attached to or ancillary to the shunt or other system described above.

The controller 202 is a processing system for controlling the operations as noted herein. In varying embodiments, the controller 202 can include a microcontroller unit, analog circuitry for signal conditioning, as well as analog to digital converters (ADC) used for powering and controlling the measurement array 204. In other embodiments, the controller 202 can include digital circuitry.

FIG. 22 illustrates one embodiment of the measurement array 204. In this embodiment, the array 204 includes a first temperature sensor 220 and a second temperature sensor 222. In this embodiment, the array 204 includes a heating element 224. For example, the sensors 220, 222 can be any suitable device or devices for sensing temperature, including but not limited to thermistors, resistive temperature detectors, or thermocouples. The heating element 224 can be any suitable element or elements that generate heat, including but not limited to a heat generating thermistor, inductive heating circuitry, resistive heating elements, or other devices or methods of applying heat. The sensors 220, 222 and heating element 224 can interact with or in response to commands from the controller 202.

With reference back to FIG. 21, the transmitter 206 can be any suitable device or devices for transmitting or otherwise communicating with the receiver 210. The data transmitter consists of driving circuitry to convert the data measurements taken and processed by the controller to be converted into wireless communication. This may use Bluetooth® or Wi-Fi frequencies, MICS band frequencies, Near Field Communication (NFC), or other available frequency bands and communication protocols to communicate. It may use frequency or amplitude shift keying as a communication protocol and may also include hardware or software safeguards to encrypt the signal and restrict access to sensitive patient info.

The power system 208 can be any system capable of storing and providing power to the elements of the system 200, including but not limited to the controller 202, the measurement array 204 and the transmitter 206. The power system may be comprised of a rechargeable battery, boost or buck converters, amplifiers, switches, and any other circuitry required to deliver power from the battery to the controller and measurement array. It can, in embodiments, also include a receiver for obtaining wirelessly delivered power. This may be comprised of inductive coils or an RF to DC converter allowing power to either be transmitted via inductive transformer or radio frequency methods, by way of example.

Receiver 210 can be external to the other elements of the system 200, in examples. For example, the receiver 210 can be a stand alone device with an installed “app” such as a phone or tablet, or may include multiple devices connected in an internet of things (IoT) to allow data to be monitored from multiple locations. It may also be a specifically engineered device containing its own power system, a built in display, and wireless connectivity. The receiver 210 can communicates with the device 200 when implanted, through any suitable protocol(s) and may also use Bluetooth ® and/or Wi-Fi connectivity to allow medical professionals to monitor patient data from any location.

The system 200 can be embedded within a shunt system or ancillary thereto. For example, in one embodiment the system 200 can be embedded with any of the systems of FIGS. 9-11.

The system 200 can perform processing operations by the measurement array 204 in response to the controller 202. In one embodiment, the measurement array 204 measures temperature of the fluid in two or more different points, for example using the sensors 220 and 222 of FIG. 22. The first measurement, acquired by the sensor 220, is taken as a reference. The heating element 224 applies a known amount of heat to the fluid. After the fluid travels within the catheter network, the second measurement, is acquired by the sensor 222.

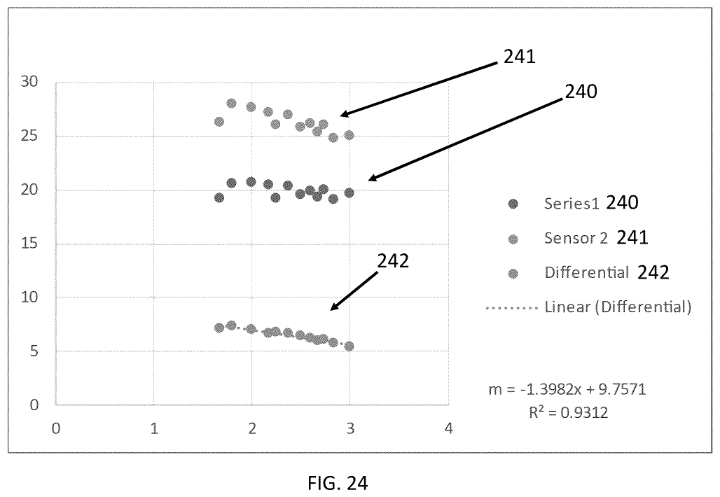

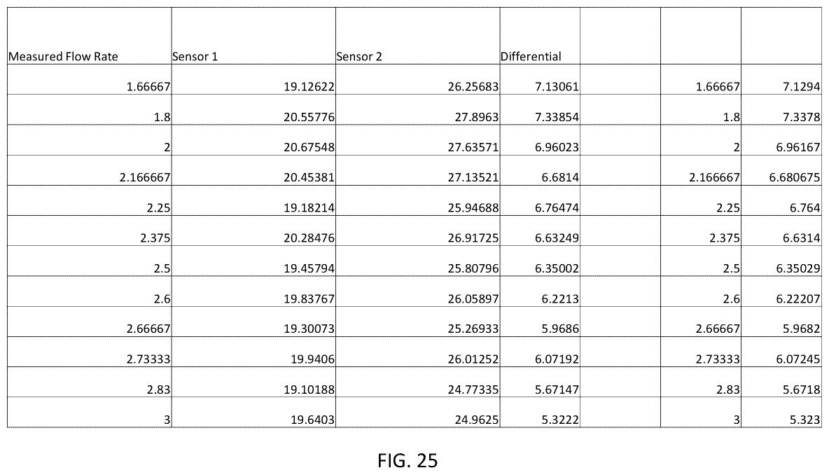

Herein, the controller and/or processor 202 receives both the first temperature value from sensor 220 and the second temperature value from the sensor 222. The controller and/or processor 202 can therein determine the flow rate of the fluid using the collected data. One embodiment provides for maintaining a constant temperature differential between the two sensors 220 and 222 and determining an amount of power required to maintain this differential, the power being the power used by the heating element 224. This power can be used to determine the mass-flow of the fluid. According to embodiments of the current invention, a faster flowing body fluid, such as CSF, will transfer or otherwise extract more heat from the two temperature sensors, resulting in a smaller temperature differential between the two temperature sensors. The exact amount of temperature differential can be dependent on the configuration of the device and location of each temperature sensor, as well as the materials it is made out of. By way of example, heat flow can be affected by the thermal conductivity of the material the sensors are made out of and the surface area that is exposed to the body fluid. The specific equation for a given temperature sensor can be derived empirically by measuring the temperature differential between the two (or more) temperature sensors for known flow rates. An example of this is shown in FIG. 24. The graph in FIG. 24 demonstrates the method of empirically deriving an equation relating flow rate to temperature differential. The first two series of data 240, 241 represent the raw data from the temperature sensors. The temperature from the first sensor 240 sets a baseline for the overall fluid temperature. The temperature from the second sensor 241 represents the fluid that has been heated by the heating element. The third dataset 242 is the difference in temperature between the sensors. This data shows a negative linear relationship between flow rate and temperature differential. It also demonstrates that this relationship is independent of the ambient temperature of the fluid. (The data populating the chart in FIG. 24 is presented in FIG. 25.)

The temperature sensors and heating element can be placed in contact, such as direct contact, with the fluid, or placed outside the tube (e.g. canula) provided that the tube (e.g., canula) has sufficient thermal conductivity to allow the temperature sensors to measure the fluid temperature. In an embodiment, a heating element can be wrapped around a small length of metal pipe or other tubing to heat the body fluid with thermistors in contact with the fluid on either side, then covering the entire device (including the two temperature sensors and the heating element) in a biocompatible material to improve efficacy and safety, and to prevent leakage.

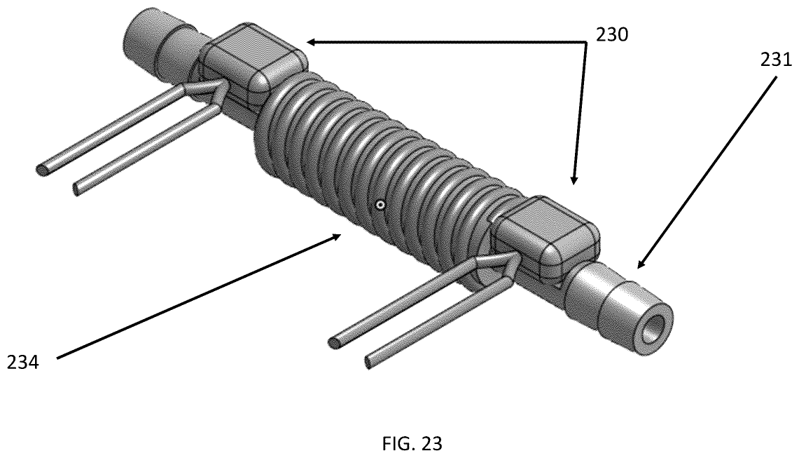

For example, as shown in FIG. 23, a tube 231 is shown wherein the at least two temperate sensors 230 measure body fluid within the tube, and wherein one or more heating element 234 is disposed between the temperature sensors.

In embodiments, biocompatible materials that can be used in the present invention, such as the device covering, include but are not limited to: polyimide, silicon, PEEK, acrylic, and others as would be understood by one of ordinary skill in the art.

Tube or tubing as used herein, can include but is not limited to a shunt, a VP shunt, a catheter, a cannula, or a pipe.

In another embodiment, a constant amount of power is used to supply the heating element 224 and the temperature differential is measured between the sensor 220 and sensor 222. In aspects, the amount of mass flowing past the heating element is directly proportional to the heat removed from the element, meaning that by measuring this change in heat, the mass flow rate can be calculated. According to embodiments of the present invention, a slower flow of body fluid can lead to less heat transfer and consequently require less power to the heating element to maintain a constant heat differential. According to embodiments of the current invention, a higher flow rate can lead to more heat transferred, resulting in higher power requirements on the device, including the heating element. These changes in temperature differential and/or measurements of the amount of power necessary to maintain a constant temperature differential are used to determine a flow rate of the body fluid.

In embodiments, the mass of fluid is measured rather than the volume. However, by way of example only, because body fluid, such as CSF, has only minor variations in density, the volumetric flow rate can also be calculated by dividing the mass by the density.

The power for both the temperature measurement as well as any driving circuitry or signal conditioning can be controlled by the controller and/or a processor 202, including for example a master control unit. In varying embodiments, the controller 202 operates an analog to digital converter required to convert the signal to a digital number and control the process of transmitting this information via the wireless communication system transmitter 206.

In further embodiments, the power system 208 can operate fully or at least partially independently and supply power to the controller 202. A master control unit of the controller 202 may also control power to the other subsystems to maximize efficiency and minimize wasted power from the power system 208.

In embodiments, data transmission may be initiated either in the receiver via a trigger signal, or it may originate in the implanted device. For example, a pull signal may originate from the receiver 210, notifying the transmitter 206 to commence data transmission. In examples, this embodiment minimizes power consumption at the transmitter 206, preventing the transmitter 206 from needlessly transmitting output when the receiver 210 is not ready for reception.

In embodiments, a communication keying protocol (e.g., amplitude and/or frequency shift keying) can be selected to maximize device security and minimize power requirements. The data could be encrypted, de-identified, or both to maximize security of patient data. It is recognized the controller 202 or other devices may include security enhancements to the data for insuring patient safety and efficacy and security of data transmission.

The receiver system may consist of one or several devices as necessary. In aspects, one device would receive the signal directly from the implanted device and may also perform additional processing and/or postprocessing calculations as necessary. The device may then connect to other devices either by Bluetooth® or Wi-Fi and display data to medical professionals or the user/wearer so problems can be intercepted as early as possible. This display may also include options for communication between patients and doctors allowing patients to report symptoms or schedule follow-up meetings as desired.

In aspects, the data can be protected by removing all personal information from it and instead associating the sensor data with a unique number for each patient. This could then be further encrypted to prevent any breach of patient confidentiality.

According to embodiments of the current invention, the device and method of fluid flow rate measurement are intended for use with CSF, but the device and method may also be used to measure any body fluid with known thermal conductivity and density, which can include, but is not limited to, blood, digestive fluid, or any other body fluid as needed. According to embodiments of the current invention, the device and method can also be used to measure the flow rate of gases of known composition.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

1. A flow meter for determining a flow rate of body fluid through a tube, the flow meter comprising the following:
a first temperature sensor measuring a first temperature of the body fluid at a first position along the tube, wherein the first temperature sensor is implanted in a human or animal body;
a heating element applying heat to the body fluid at a second position along the tube, wherein the heating element applies heat to the body fluid after the body fluid passes the first position, and wherein the heating element is implanted in the human or animal body;
a second temperature sensor measuring a second temperature of the body fluid at a third position along the tube, wherein the second temperature sensor is located along the tube at a location after the body fluid passes the second position, and wherein the second temperature sensor is implanted in the human or animal body;
a controller configured to receive the first temperature, the second temperature, and a heat value relating to the heat applied to the body fluid by the heating element; and
a processing device configured to calculate a flow rate of the body fluid through the tube based at least on the first temperature, the second temperature, and the heat value.

2. The flow meter of claim 1, further comprising:
a transmitter receiving the flow rate and transmitting the flow rate to a receiver.

3. The flow meter of claim 2, wherein the transmitter transmits the flow rate using at least one wireless transmission technology.

4. The flow meter of claim 1, further comprising:
at least one power system in communication with the controller such that the heat element is operative to apply the heat in response to a power control command from the controller.

5. The flow meter of claim 4, wherein the power system includes at least one battery for storing an electrical charge therein.

6. The flow meter of claim 4, wherein the power system includes at least one charging element for generating an electrical charge.

7. The flow meter of claim 6, wherein the at least one charging element includes a Near-field Communication component.

8. The flow meter of claim 1, further comprising:
at least one receiver in wireless communication with the controller for generating at least one output display based on the flow rate.

9. The flow meter of claim 1, wherein the heat element includes at least one of: a heat generating thermistor, inductive heating circuitry, and resistive heating elements.

10. The flow meter of claim 1, wherein the first temperature sensor and the second temperature sensor each include at least one of: a thermistor, a resistive temperature detector, a semiconducting temperature sensor, and a thermocouple.

11. The flow meter of claim 1, wherein the calculating of the flow rate of the body fluid is based on maintaining a constant temperature differential between the first temperature and the second temperature, such that a power requirement for the heating element to maintain the constant temperature differential is provided for calculating the flow rate.

12. The flow meter of claim 1, wherein the calculating of the flow rate of the body is based on using a defined amount of power to power the heating element and measuring a temperature differential between the first temperature and the second temperature, such that the defined amount of power is provided for calculating the flow rate.

13. The flow meter of claim 1, wherein the tube is a shunt, wherein the flow meter is connected to the shunt and a bi-compartmental valve, wherein the bi-compartmental valve acts to modulate the flow rate of the body fluid from a first location along the shunt, to a second location along the shunt.

14. The flow meter of claim 13, wherein the first location is one or more patient ventricle.

15. The flow meter of claim 13, wherein the second location is a fluid receiver.

16. The flow meter of claim 13, wherein the bi-compartmental valve comprises a reservoir for transcutaneous tapping.

17. The flow meter of claim 13, wherein the shunt is a ventriculoperitoneal shunt.

18. The flow meter of claim 13, wherein the shunt is a ventriculoperitoneal shunt for treatment of hydrocephalus.

19. The flow meter of claim 1, wherein the tube is a catheter.

20. The flow meter of claim 19, wherein the catheter is a ventricular catheter.

21. The flow meter of claim 19, wherein the catheter is a peritoneal catheter.
